(19) United States
(12) Patent Application Publication
(10) Pub. No.: US 2012/0095028 A1
(43) Pub. Date: Apr. 19, 2012

(54) 3-OXA-7-AZABICYCLO[3.3.1]NONANES

(75) Inventors: Michael Paul DeNinno, Encinitas, CA (US); Kentaro Futatsugi, Niantic, CT (US); Bruce Allen Lefker, Gales Ferry, CT (US); Vincent Mascitti, Groton, CT (US); Kim Francis McClure, Mystic, CT (US); Michael John Munchhof, Salem, CT (US)

(73) Assignee: Pfizer Inc.

(21) Appl. No.: 13/257,384

(22) PCT Filed: Mar. 4, 2010
(86) PCT No.: PCT/IB2010/050945

§ 371 Date: Sep. 19, 2011

Related U.S. Application Data
(60) Provisional application No. 61/162,000, filed on Mar. 20, 2009.
(60) Provisional application No. 61/173,693, filed on Apr. 29, 2009.

(51) Int. Cl. A61K 31/519; A61P 13/00; A61P 3/00; A61P 3/04; A61P 3/06; A61P 3/10; A61P 9/00; A61P 19/02; A61P 19/10; A61P 9/12; A61P 13/12; A61P 15/00; A61P 9/10; A61P 15/10; A61P 17/00; A61P 25/28; A61P 25/18; A61P 1/00; C07D 471/04

(52) U.S. Cl. 514/264.1; 544/280

(57) ABSTRACT

Compounds of Formula (I) that modulate the activity of the G-protein-coupled receptor GPR119 and their uses in the treatment of diseases linked to the modulation of the G-protein-coupled receptor GPR119 in animals are described herein.

### FIELD OF THE INVENTION

The present invention relates to a new class of 3-oxa-7-azabicyclo[3.3.1]nonanes, pharmaceutical compositions containing these compounds, and their use to modulate the activity of the G-protein-coupled receptor, GPR119.

### BACKGROUND

Diabetes mellitus are disorders in which high levels of blood glucose occur as a consequence of abnormal glucose homeostasis. The most common forms of diabetes mellitus are Type I (also referred to as insulin-dependent diabetes mellitus) and Type II diabetes (also referred to as non-insulin-dependent diabetes mellitus). Type II diabetes, accounting for roughly 90% of all diabetic cases, is a serious progressive disease that results in microvascular complications (including retinopathy, neuropathy and nephropathy) as well as macrovascular complications (including accelerated atherosclerosis, coronary heart disease and stroke).

Currently, there is no cure for diabetes. Standard treatments for the disease are limited, and focus on controlling blood glucose levels to minimize or delay complications. Current treatments target either insulin resistance (metformin, thiazolidinediones, or insulin release from beta cells (sulphonylureas, exanatide). Sulphonylureas and other compounds that act via depolarization of the beta cell promote hypoglycemia as they stimulate insulin secretion independent of circulating glucose concentrations. One approved drug, exanatide, stimulates insulin secretion only in the presence of high glucose, but must be injected due to a lack of oral bioavailablity. Sitagliptin, a dipeptidyl peptidase IV inhibitor, is a new drug that increases blood levels of incretin hormones, which can increase insulin secretion, reduce glucagon secretion and have other less well characterized effects. However, sitagliptin and other dipeptidyl peptidases IV inhibitors may also influence the tissue levels of other hormones and peptides, and the long-term consequences of this broader effect have not been fully investigated.

In Type II diabetes, muscle, fat and liver cells fail to respond normally to insulin. This condition (insulin resistance) may be due to reduced numbers of cellular insulin receptors, disruption of cellular signaling pathways, or both. At first, the beta cells compensate for insulin resistance by increasing insulin output. Eventually, however, the beta cells become unable to produce sufficient insulin to maintain normal glucose levels (euglycemia), indicating progression to Type II diabetes.

In Type II diabetes, fasting hyperglycemia occurs due to insulin resistance combined with beta cell dysfunction. There are two aspects of beta cell defect dysfunction: 1) increased basal insulin release (occurring at low, non-stimulatory glucose concentrations). This is observed in obese, insulin-resistant pre-diabetic stages as well as in Type II diabetes, and 2) in response to a hyperglycemic challenge, a failure to increase insulin release above the already elevated basal level. This does not occur in pre-diabetic stages and may signal the transition from normo-glycemic insulin-resistant states to frank Type II diabetes. Current therapies to treat the latter aspect include inhibitors of the beta-cell ATP-sensitive potassium channel to trigger the release of endogenous insulin stores, and administration of exogenous insulin. Neither achieves accurate normalization of blood glucose levels and both carry the risk of eliciting hypoglycemia.

Thus, there has been great interest in the discovery of agents that function in a glucose-dependent manner. Physiological signaling pathways which function in this way are well known, including gut peptides GLP-1 and GIP. These hormones signal via cognate G-protein coupled receptors to stimulate production of cAMP in pancreatic beta-cells. Increased cAMP apparently does not result in stimulation of insulin release during the fasting or pre-prandial state. However, a number of biochemical targets of cAMP, including the ATP-sensitive potassium channel, voltage-sensitive potassium channels and the exocytotic machinery, are modulated such that insulin secretion due to postprandial glucose stimulation is significantly enhanced. Therefore, agonist modulators of novel, similarly functioning, beta-cell GPCRs, including GPR119, would also stimulate the release of endogenous insulin and promote normalization of glucose levels in Type II diabetes patients. It has also been shown that increased cAMP, for example as a result of GLP-1 stimulation, promotes beta-cell proliferation, inhibits beta-cell death and thus improves islet mass. This positive effect on beta-cell mass should be beneficial in Type II diabetes where insufficient insulin is produced.

It is well known that metabolic diseases have negative effects on other physiological systems and there is often co-occurrence of multiple disease states (e.g. type I diabetes, type II diabetes, inadequate glucose tolerance, insulin resistance, hyperglycemia, hyperlipidemia, hypertriglyceridemia, hypercholesterolemia, dyslipidemia, obesity or cardiovascular disease in “Syndrome X”) or secondary diseases which occur secondary to diabetes such as kidney disease, and peripheral neuropathy. Thus, treatment of the diabetic condition should be of benefit to such interconnected disease states.

### SUMMARY OF THE INVENTION

In accordance with the present invention, a new class of 3-oxa-7-azabicyclo[3.3.1]nonanes has been discovered. These compounds may be represented by Formula I, as shown below:

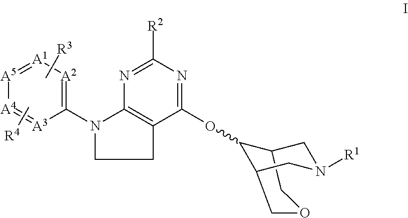

in which
R¹ is represented by CO—O—R⁵ or a substituted pyrimidine represented by the following formula

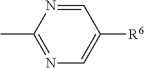

R² is represented by hydrogen or methyl;
R³ is represented by a subsituent selected from the group consisting of hydrogen, halogen, cyano, CF₃, OCF₃, C₁-C₅ alkoxy, and C₁-C₅ alkyl;
R⁴ is absent, or is represented by SO₂—R⁷, CO—NR⁸R⁹, tetrazole, C₁-C₅ alkyl, NH₂, —NH—C₁-C₅ alkyl, —NH—CO—C₁-C₅ alkyl, NH—(CH₂)₂—OH;
R⁵ is represented by C₁-C₅ alkyl, or C₃-C₆cycloalkyl;
R⁶ is represented by CF₃, C₁-C₅ alkyl, halogen, cyano, or C₃-C₆cycloalkyl;
R⁷ is represented by C₃-C₆cycloalkyl, C₁-C₅ alkyl, NH₂, or (CH₂)₂—OH;
R⁸ is represented by hydrogen or C₁-C₅ alkyl,
R⁹ is represented by hydrogen, C₁-C₅ alkyl, C₃-C₆ cycloalkyl, CH₂—CH₂—OH, CH₂—CH₂—O—CH₃, CH₂—CH₂—CH₂—O—CH₃, CH₂—CH₂—CH₂—OH, 3-oxetanyl, 3-hydroxycyclobutyl,
A¹, A², A³, A⁴, and A⁵ are each independently represented by CH, N-oxide, or N;
with the proviso that:
1) no more than 2 of A¹, A², A³, A⁴, and A⁵ are represented by N;
2) no more than 1 of A¹, A², A³, A⁴, and A⁵ are represented by N-oxide;
or a pharmaceutically acceptable salt thereof.

The compounds of Formula I modulate the activity of the G-protein-coupled receptor. More specifically the compounds modulate GPR119. As such, said compounds are useful for the treatment of diseases, such as diabetes, in which the activity of GPR119 contributes to the pathology or symptoms of the disease. Examples of such conditions include hyperlipidemia, type I diabetes, type II diabetes mellitus, idiopathic type I diabetes (Type Ib), latent autoimmune diabetes in adults (LADA), early-onset type 2 diabetes (EOD), youth-onset atypical diabetes (YOAD), maturity onset diabetes of the young (MODY), malnutrition-related diabetes, gestational diabetes, coronary heart disease, ischemic stroke, restenosis after angioplasty, peripheral vascular disease, intermittent claudication, myocardial infarction (e.g. necrosis and apoptosis), dyslipidemia, post-prandial lipemia, conditions of impaired glucose tolerance (IGT), conditions of impaired fasting plasma glucose, metabolic acidosis, ketosis, arthritis, obesity, osteoporosis, hypertension, congestive heart failure, left ventricular hypertrophy, peripheral arterial disease, diabetic retinopathy, macular degeneration, cataract, diabetic nephropathy, glomerulosclerosis, chronic renal failure, diabetic neuropathy, metabolic syndrome, syndrome X, premenstrual syndrome, coronary heart disease, angina pectoris, thrombosis, atherosclerosis, transient ischemic attacks, stroke, vascular restenosis, hyperglycemia, hyperinsulinemia, hyperlipidemia, hypertrygliceridemia, insulin resistance, impaired glucose metabolism, conditions of impaired glucose tolerance, conditions of impaired fasting plasma glucose, obesity, erectile dysfunction, skin and connective tissue disorders, foot ulcerations and ulcerative colitis, endothelial dysfunction and impaired vascular compliance. The compounds may be used to treat neurological disorders such as Alzheimer's, schizophrenia, and impaired cognition. The compounds will also be beneficial in gastrointestinal illnesses such as inflammatory bowel disease, ulcerative colitis, Crohn's disease, irritable bowel syndrome, etc. As noted above the compounds may also be used to stimulate weight loss in obese patients, especially those afflicted with diabetes.

A further embodiment of the invention is directed to pharmaceutical compositions containing a compound of Formula I. Such formulations will typically contain a compound of Formula I in admixture with at least one pharmaceutically acceptable excipient. Such formulations may also contain at least one additional pharmaceutical agent (described herein). Examples of such agents include anti-obesity agents and/or anti-diabetic agents (described herein below). Additional aspects of the invention relate to the use of the compounds of Formula I in the preparation of medicaments for the treatment of diabetes and related conditions as described herein.

### DETAILED DESCRIPTION OF THE INVENTION

The headings within this document are only being utilized to expedite its review by the reader. They should not be construed as limiting the invention or claims in any manner.

### Definitions and Exemplification

As used throughout this application, including the claims, the following terms have the meanings defined below, unless specifically indicated otherwise. The plural and singular should be treated as interchangeable, other than the indication of number:
a. “halogen” refers to a chlorine, fluorine, iodine, or bromine atom.
b. “C₁-C₅ alkyl” refers to a branched or straight chained alkyl group containing from 1 to 5 carbon atoms, such as methyl, ethyl, n-propyl, isopropyl, n-butyl, isobutyl, pentyl, etc.
c. “C₁-C₅ alkoxy” refers to a straight or branched chain alkoxy group containing from 1 to 5 carbon atoms, such as methoxy, ethoxy, n-propoxy, isopropoxy, n-butoxy, isobutoxy, pentoxy, etc.
d. “C₃-C₆ cycloalkyl” refers to a nonaromatic ring that is fully hydrogenated and exists as a single ring. Examples of such carbocyclic rings include cyclopropyl, cyclobutyl, cyclopentyl, and cyclohexyl,
e. “therapeutically effective amount” means an amount of a compound of the present invention that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.
f. “patient” refers to warm blooded animals such as, for example, guinea pigs, mice, rats, gerbils, cats, rabbits, dogs, monkeys, chimpanzees, and humans.
g. “treat” refers to the ability of the compounds to either relieve, alleviate, or slow the progression of the patient's disease (or condition) or any tissue damage associated with the disease.
h. “the terms “modulated”, “modulating”, or “modulate(s)”, as used herein, unless otherwise indicated, refers to the activation of the G-protein-coupled receptor GPR119 with compounds of the present invention.
i. “pharmaceutically acceptable” indicates that the substance or composition must be compatible chemically and/or toxicologically, with the other ingredients comprising a formulation, and/or the mammal being treated therewith.
j. “salts” is intended to refer to pharmaceutically acceptable salts and to salts suitable for use in industrial processes, such as the preparation of the compound.
k. “pharmaceutically acceptable salts” is intended to refer to either pharmaceutically acceptable acid addition salts” or “pharmaceutically acceptable basic addition salts” depending upon actual structure of the compound.
l. “pharmaceutically acceptable acid addition salts” is intended to apply to any non-toxic organic or inorganic acid addition salt of the base compounds represented by Formula I or any of its intermediates. Illustrative inorganic acids which form suitable salts include hydrochloric, hydrobromic, sulphuric, and phosphoric acid and acid metal salts such as sodium monohydrogen orthophosphate, and potassium hydrogen sulfate. Illustrative organic acids, which form suitable salts include the mono-, di-, and tricarboxylic acids. Illustrative of such acids are for example, acetic, glycolic, lactic, pyruvic, malonic, succinic, glutaric, fumaric, malic, artaric, citric, ascorbic, maleic, hydroxymaleic, benzoic, hydroxy-benzoic, phenylacetic, cinnamic, salicylic, 2-phenoxybenzoic, p-toluenesulfonic acid, and sulfonic acids such as methane sulfonic acid and 2-hydroxyethane sulfonic acid. Such salts can exist in either a hydrated or substantially anhydrous form. In general, the acid addition salts of these compounds are soluble in water and various hydrophilic organic solvents.
m. “pharmaceutically acceptable basic addition salts” is intended to apply to any non-toxic organic or inorganic basic addition salts of the compounds represented by Formula I, or any of its intermediates. Illustrative bases which form suitable salts include alkali metal or alkaline-earth metal hydroxides such as sodium, potassium, calcium, magnesium, or barium hydroxides; ammonia, and aliphatic, alicyclic, or aromatic organic amines such as methylamine, dimethylamine, trimethylamine, and picoline.
n. “compound of Formula I”, “compounds of the invention”, and “compounds” are used interchangeably throughout the application and should be treated as synonoms.
   “isomer” means “stereoisomer” and “geometric isomer” as defined below.
o. “stereoisomer” means compounds that possess one or more chiral centers and each center may exist in the R or S configuration. Stereoisomers includes all diastereomeric, enantiomeric and epimeric forms as well as racemates and mixtures thereof.
p. “geometric isomer” means compounds that may exist in cis, trans, anti, syn, entgegen (E), and zusammen (Z) forms as well as mixtures thereof.

Certain of the compounds of the formula (I) may exist as geometric isomers. The compounds of the formula (I) may possess one or more asymmetric centers, thus existing as two, or more, stereoisomeric forms. The present invention includes all the individual stereoisomers and geometric isomers of the compounds of formula (I) and mixtures thereof. Individual enantiomers can be obtained by chiral separation or using the relevant enantiomer in the synthesis.

In addition, the compounds of the present invention can exist in unsolvated as well as solvated forms with pharmaceutically acceptable solvents such as water, ethanol, and the like. In general, the solvated forms are considered equivalent to the unsolvated forms for the purposes of the present invention. The compounds may also exist in one or more crystalline states, i.e. polymorphs, or they may exist as amorphous solids. All such forms are encompassed by the claims.

All of the compounds of Formula I contain an azabicyclo-nonane ring bonded to a pyrrolo-pyrimidine ring via an ether linkage as depicted below. This azabicyclo-nonane will exist as a geometric isomer and may be present as either the syn or anti isomer as depicted below.

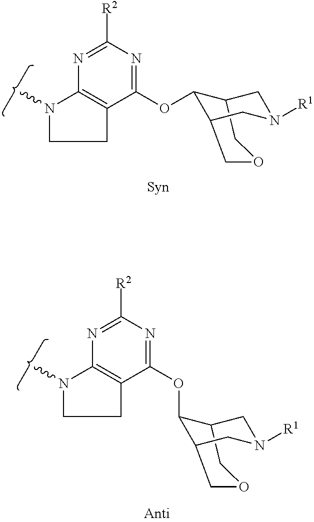

All of the compounds of Formula I contain a phenyl ring or a nitrogen containing aromatic as depicted below:

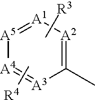

A¹-A⁵ may represent up to two nitrogen atoms and the remainder will be CH. Thus, this aromatic ring may represent, for example, phenyl, pyridyl, pyrimidinyl, pyridazinyl, or pyrazinyl. R³ may be hydrogen, or one of the substituents specified above. When R³ is not hydrogen, it may represent up to 2 substituents that may be bonded to any carbon atom of the ring (with the exception of the carbon bonded to the pyrrolo-pyrimidine moiety). R⁴ may be present, or absent, and if present may be bonded to any carbon atom on the ring (with the exception of the carbon bonded to the pyrrolo-pyrimidine moiety).

Additionally one of A¹-A⁵ may represent an N-oxide moiety. In any situation in which the aryl moiety represented by A¹-A⁵ is substituted, then the relevant carbon atom will represent CR³ or CR⁴, not CH; as is readily apparent to one skilled in the art.

Examples of such nitrogen containing rings include:

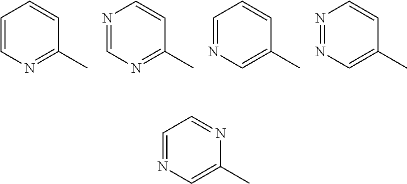

In more specific embodiments:
a) R¹ is C(O)—O—C₁-C₅alkyl, and R² is hydrogen;
b) R¹ is C(O)—O—C₁-C₅alkyl, R² is hydrogen, and A¹-A⁵ forms a phenyl ring;
c) R¹ is C(O)—O—C₁-C₅alkyl, R² is hydrogen, A¹-A⁵ forms a phenyl ring, and R³ is fluoro;
d) R¹ is C(O)—O—C₁-C₅alkyl, R² is hydrogen, A¹-A⁵ forms a phenyl ring, R³ is fluoro, R⁴ is present and is represented by —SO₂—R⁷ or —NH—CO—C₁-C₅ alkyl;
e) R¹ is C(O)—O—C₁-C₅alkyl, R² is hydrogen, A¹-A⁵ forms a phenyl ring, R³ is fluoro, R⁴ is present and is represented by —SO₂—R⁷;
f) R¹ is C(O)—O—C₁-C₅alkyl, R² is hydrogen, A¹-A⁵ forms a phenyl ring, R³ is fluoro, R⁴ is present and is represented by NH—(CH₂)₂—OH;
g) R¹ is C(O)—O—C₁-C₅alkyl, R² is hydrogen, A¹-A⁵ forms a phenyl ring, R³ is fluoro and cyano;
h) R¹ is C(O)—O—C₁-C₅alkyl, R² is hydrogen, and A¹-A⁵ form a pyridyl ring;
i) R¹ is C(O)—O—C₁-C₅alkyl, R² is hydrogen, A¹-A⁵ forms a pyridyl ring and R³ is fluoro;
j) R¹ is C(O)—O—C₁-C₅alkyl, R² is hydrogen, A¹-A⁵ forms a pyridyl ring, R³ is fluoro, and R⁴ is present and is represented by —SO₂—R⁷ or —NH—CO—C₁-C₅ alkyl,
k) R¹ is C(O)—O—C₁-C₅alkyl, R² is hydrogen, A¹-A⁵ forms a pyridyl ring, R³ is fluoro, R⁴ is present and is represented by —SO₂—R⁷;
l) R¹ is C(O)—O—C₁-C₅alkyl, R² is hydrogen, A¹-A⁵ forms a pyridyl ring, R³ is fluoro, R⁴ is present and is represented by NH—(CH₂)₂—OH, and;
m) R¹ is C(O)—O—C₁-C₅alkyl, R² is hydrogen, A¹-A⁵ forms a pyridyl ring, R³ is fluoro and cyano.

### Synthesis

Compounds of the invention may be synthesized by synthetic routes that include processes analogous to those well-known in the chemical arts, particularly in light of the description contained herein. The starting materials are generally available from commercial sources such as Aldrich Chemicals (Milwaukee, Wis.) or are readily prepared using methods known to those skilled in the art (e.g., prepared by methods generally described in Louis F. Fieser and Mary Fieser, Reagents for Organic Synthesis, v. 1-19, Wiley, New York (1967-1999 ed.), or Beilsteins Handbuch der organischen Chemie, 4, Aufl. ed. Springer-Verlag, Berlin, including supplements (also available via the Beilstein online database).

For illustrative purposes, the reaction schemes depicted below provide potential routes for synthesizing the compounds of the present invention as well as key intermediates. For a more detailed description of the individual reaction steps, see the Examples section below. Those skilled in the art will appreciate that other synthetic routes may be used to synthesize the inventive compounds. Although specific starting materials and reagents are depicted in the schemes and discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

The compounds of Formula I can be prepared using methods analogously known in the art for the production of ethers. The reader's attention is focused to texts such as: 1) Hughes, D. L.; Organic Reactions 1992, 42 Hoboken, N.J., United States; 2) Tikad, A.; Routier, S.; Akssira, M.; Leger, J.-M. I; Jarry, C.; Guillaumet, G. Synlett 2006, 12, 1938-42; and 3) Loksha, Y. M.; Globisch, D.; Pedersen, E. B.; La Colla, P.; Collu, G.; Loddo, R. J. Het. Chem. 2008, 45, 1161-6 which describe such reactions in greater detail.

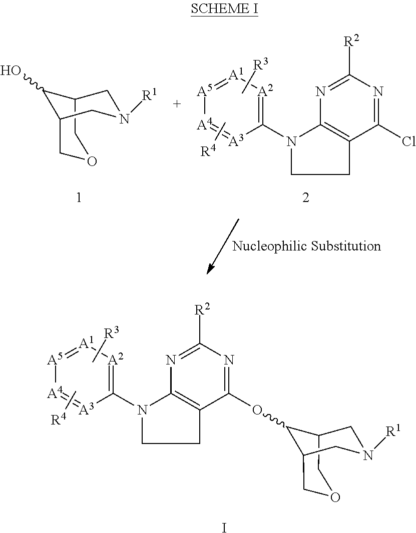

As depicted above, one of the starting materials is a 3-oxa-7-azabicyclo[3.3.1]nonanol as described by structure 1. R¹ will typically be represented by the same substituent as is desired in the final product. Reaction Scheme II, hereinafter, teaches a method for the production of such alcohols.

The other starting material is the chloride of structure 2. R², R³, R⁴ and A¹-A⁵ will typically be represented by the same moiety as desired in the final product. These compounds are also known in the art. Methods for their preparation have been described in WO 2008/008895 and are also analogously known in the art.

The nucleophilic reaction is carried out as is known in the art. Typically approximately equivalent amounts of the alcohol of structure 1 and the chloride of structure 2 are contacted in a polar aprotic solvent such as dioxane, tetrahydrofuran, dimethylsulfoxide, or dimethylformamide. The reactants are then contacted with a base such as sodium hydroxide, sodium hexamethyldisilazide, potassium hexamethyldisilazide, or potassium t-amyloxide in a quantity ranging from 0.9 to 10 equivalents at a temperature range of room temperature to 110° C. Typically, the reaction will be allowed to proceed for a period of time ranging from 15 minutes to 14 hours under an inert atmosphere.

After the reaction is completed the desired compound of Formula I may be recovered and isolated as known in the art. It may be recovered by evaporation, extraction, etc. as is known in the art. It may optionally be purified by chromatography, recrystallization, distillation, or other techniques known in the art prior.

As is also readily apparent to one skilled in the art, many of the substituents represented by R¹ and R⁴ may be manipulated after the core of Formula I is produced. Such variations are well known to those skilled in the art and should be considered part of the invention.

Reaction Scheme II, immediately below, teaches a method for the production of the azabicyclo-nonanes as described by structure 1 above. Compound #1 of Reaction Scheme #2 is known in the art. It's synthesis is taught in Arjunan, P.; Berlin, K. D.; Barnes C. L.; Van der Helm, D. J. Org. Chem., 1981, 46 (16), 3196-3204.

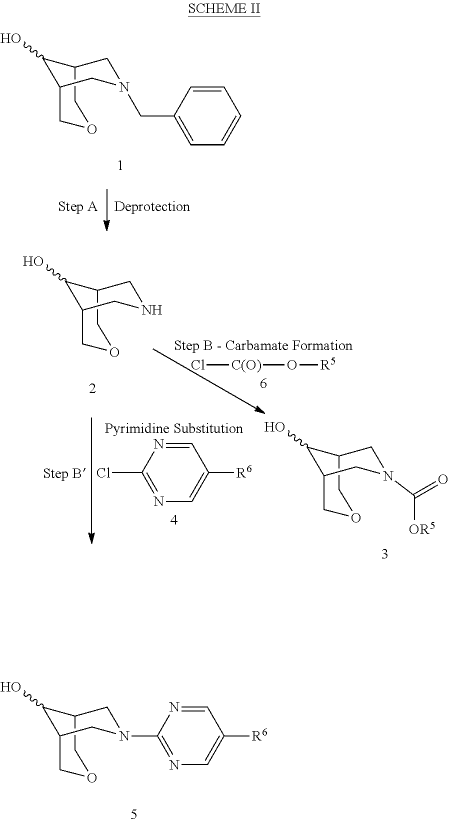

As shown above, the initial step in the reaction is to remove the benzyl protecting group from structure 1. This can be accomplished via hydrogenolysis to give compound 2. Typical conditions for this reaction include utilizing hydrogen and a palladium catalyst including 5-20% palladium on carbon or 10-20% palladium hydroxide. A typical solvent for this reaction is ethanol, methanol, tetrahydrofuran or ethyl acetate.

If a pyrimidine substituent is desired in the final product, then structure 5 may be formed via the addition of compound 2 to an appropriately substituted 2-chloropyrimidine as depicted by structure 4 in the presence of a base such as cesium carbonate or diisopropylethylamine in a protic solvent such as ethanol or methanol, or a polar aprotic solvent such as 1,4-dioxane, tetrahydrofuran, dimethylformamide or dimethylsulfoxide. These reactions can be conducted at temperatures ranging from room temperature to 110° C. Alternatively, compounds of structure 2 and structure 4 can be heated together in the presence of base such as diisopropylethylamine without solvent, or where compound 2 is used in excess without base or solvent.

If a carbamate substituent is desired in the final product then equivalent amounts the alkyl haloformate formate of structure 6 is contacted with the compound of structure 2 in the presence of a base such as diisopropylethylamine, triethylamine or pyridine in dichloromethane or chloroform. Alternatively, compounds of structure 3 can formed from compounds of structure 2 via the use of dialkyldicarbonates such as di-tert-butyl dicarbonate (BOC anhydride) or di-isopropyl dicarbonate in the presence of amine bases such as diisopropylethylamine, pyridine, 2,6-lutidine or triethylamine in solvents such as dichloromethane, chloroform or tetrahydrofuran.

Final structure 3 or 5 (i.e. structure #1 from Reaction Scheme 1) may be isolated and purified as is known in the art. If desired, it may be subjected to a separation step to yield the desired syn or anti isomer prior to its utilization in Reaction Scheme I.

As is readily apparent to one skilled in the art, protection of remote functionality (e.g., primary or secondary amine) of intermediates may be necessary. The need for such protection will vary depending on the nature of the remote functionality and the conditions of the preparation methods. Suitable amino-protecting groups (NH-Pg) include acetyl, trifluoroacetyl, t-butoxycarbonyl (BOC), benzyloxycarbonyl (CBZ) and 9-fluorenylmethyleneoxycarbonyl (Fmoc). Similarly, a “hydroxy-protecting group” refers to a substituent of a hydroxy group that blocks or protects the hydroxy functionality. Suitable hydroxyl-protecting groups (O-Pg) include for example, allyl, acetyl, silyl, benzyl, para-methoxybenzyl, trityl, and the like. The need for such protection is readily determined by one skilled in the art. For a general description of protecting groups and their use, see T. W. Greene, Protective Groups in Organic Synthesis, John Wiley & Sons, New York, 1991.

As noted above, some of the compounds of this invention are acidic and they form salts with pharmaceutically acceptable cations. Some of the compounds of this invention are basic and form salts with pharmaceutically acceptable anions. All such salts are within the scope of this invention and they can be prepared by conventional methods such as combining the acidic and basic entities, usually in a stoichiometric ratio, in either an aqueous, non-aqueous or partially aqueous medium, as appropriate. The salts are recovered either by filtration, by precipitation with a non-solvent followed by filtration, by evaporation of the solvent, or, in the case of aqueous solutions, by lyophilization, as appropriate. The compounds are obtained in crystalline form according to procedures known in the art, such as by dissolution in an appropriate solvent(s) such as ethanol, hexanes or water/ethanol mixtures

As noted above, some of the compounds exist as isomers. These isomeric mixtures can be separated into their individual isomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereoisomers and converting (e.g., hydrolyzing) the individual diastereoisomers to the corresponding pure enantiomers. Enantiomers can also be separated by use of a chiral HPLC column. Alternatively, the specific stereoisomers may be synthesized by using an optically active starting material, by asymmetric synthesis using optically active reagents, substrates, catalysts or solvents, or by converting one stereoisomer into the other by asymmetric transformation.

The present invention also embraces isotopically-labeled compounds of the present invention which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, sulfur, fluorine, iodine, and chlorine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ¹²³I, ¹²⁵I and ³⁶Cl, respectively.

Certain isotopically-labeled compounds of the present invention (e.g., those labeled with ³H and ¹⁴C) are useful in compound and/or substrate tissue distribution assays. Certain isotopically labeled ligands including tritium, ¹⁴C, ³⁵S and ¹²⁵I could be useful in radioligand binding assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Positron emitting isotopes such as ¹⁵O, ¹³N, ¹¹C, and ¹⁸F are useful for positron emission tomography (PET) studies to examine receptor occupancy. Isotopically labeled compounds of the present invention can generally be prepared by following procedures analogous to those disclosed in the Schemes and/or in the Examples herein below, by substituting an isotopically labeled reagent for a non-isotopically labeled reagent.

Certain compounds of the present invention may exist in more than one crystal form (generally referred to as “polymorphs”). Polymorphs may be prepared by crystallization under various conditions, for example, using different solvents or different solvent mixtures for recrystallization; crystallization at different temperatures; and/or various modes of cooling, ranging from very fast to very slow cooling during crystallization. Polymorphs may also be obtained by heating or melting the compound of the present invention followed by gradual or fast cooling. The presence of polymorphs may be determined by solid probe NMR spectroscopy, IR spectroscopy, differential scanning calorimetry, powder X-ray diffraction or such other techniques.

### Medical Uses

Compounds of the present invention modulate the activity of G-protein-coupled receptor GPR1 19. As such, said compounds are useful for the prophylaxis and treatment of diseases, such as diabetes, in which the activity of GPR119 contributes to the pathology or symptoms of the disease. Consequently, another aspect of the present invention includes a method for the treatment of a metabolic disease and/or a metabolic-related disorder in an individual which comprises administering to the individual in need of such treatment a therapeutically effective amount of a compound of the invention, a salt of said compound or a pharmaceutical composition containing such compound. The metabolic diseases and metabolism-related disorders are selected from, but not limited to, hyperlipidemia, type I diabetes, type II diabetes mellitus, idiopathic type I diabetes (Type Ib), latent autoimmune diabetes in adults (LADA), early-onset type 2 diabetes (EOD), youth-onset atypical diabetes (YOAD), maturity onset diabetes of the young (MODY), malnutrition-related diabetes, gestational diabetes, coronary heart disease, ischemic stroke, restenosis after angioplasty, peripheral vascular disease, intermittent claudication, myocardial infarction (e.g. necrosis and apoptosis), dyslipidemia, post-prandial lipemia, conditions of impaired glucose tolerance (IGT), conditions of impaired fasting plasma glucose, metabolic acidosis, ketosis, arthritis, obesity, osteoporosis, hypertension, congestive heart failure, left ventricular hypertrophy, peripheral arterial disease, diabetic retinopathy, macular degeneration, cataract, diabetic nephropathy, glomerulosclerosis, chronic renal failure, diabetic neuropathy, metabolic syndrome, syndrome X, premenstrual syndrome, coronary heart disease, angina pectoris, thrombosis, atherosclerosis, myocardial infarction, transient ischemic attacks, stroke, vascular restenosis, hyperglycemia, hyperinsulinemia, hyperlipidemia, hypertrygliceridemia, insulin resistance, impaired glucose metabolism, conditions of impaired glucose tolerance, conditions of impaired fasting plasma glucose, obesity, erectile dysfunction, skin and connective tissue disorders, foot ulcerations, endothelial dysfunction, hyper apo B lipoproteinemia and impaired vascular compliance. Additionally, the compounds may be used to treat neurological disorders such as Alzheimer's, schizophrenia, and impaired cognition. The compounds will also be beneficial in gastrointestinal illnesses such as inflammatory bowel disease, ulcerative colitis, Crohn's disease, irritable bowel syndrome, etc. As noted above the compounds may also be used to stimulate weight loss in obese patients, especially those afflicted with diabetes.

In accordance with the foregoing, the present invention further provides a method for preventing or ameliorating the symptoms of any of the diseases or disorders described above in a subject in need thereof, which method comprises administering to a subject a therapeutically effective amount of a compound of the present invention. Further aspects of the invention include the preparation of medicaments for the treating diabetes and its related co-morbidities.

In order to exhibit the therapeutic properties described above, the compounds need to be administered in a quantity sufficient to modulate activation of the G-protein-coupled receptor GPR119. This amount can vary depending upon the particular disease/condition being treated, the severity of the patient's disease/condition, the patient, the particular compound being administered, the route of administration, and the presence of other underlying disease states within the patient, etc. When administered systemically, the compounds typically exhibit their effect at a dosage range of from about 0.1 mg/kg/day to about 100 mg/kg/day for any of the diseases or conditions listed above. Repetitive daily administration may be desirable and will vary according to the conditions outlined above.

The compounds of the present invention may be administered by a variety of routes. They may be administered orally. The compounds may also be administered parenterally (i.e., subcutaneously, intravenously, intramuscularly, intraperitoneally, or intrathecally), rectally, or topically.

### Co-Administration

The compounds of this invention may also be used in conjunction with other pharmaceutical agents for the treatment of the diseases, conditions and/or disorders described herein. Therefore, methods of treatment that include administering compounds of the present invention in combination with other pharmaceutical agents are also provided. Suitable pharmaceutical agents that may be used in combination with the compounds of the present invention include anti-obesity agents (including appetite suppressants), anti-diabetic agents, anti-hyperglycemic agents, lipid lowering agents, and anti-hypertensive agents.

Suitable anti-diabetic agents include an acetyl-CoA carboxylase-2 (ACC-2) inhibitor, a diacylglycerol O-acyltransferase 1 (DGAT-1) inhibitor, a phosphodiesterase (PDE)-10 inhibitor, a sulfonylurea (e.g., acetohexamide, chlorpropamide, diabinese, glibenclamide, glipizide, glyburide, glimepiride, gliclazide, glipentide, gliquidone, glisolamide, tolazamide, and tolbutamide), a meglitinide, an α-amylase inhibitor (e.g., tendamistat, trestatin and AL-3688), an a-glucoside hydrolase inhibitor (e.g., acarbose), an α-glucosidase inhibitor (e.g., adiposine, camiglibose, emiglitate, miglitol, voglibose, pradimicin-Q, and salbostatin), a PPARγ agonist (e.g., balaglitazone, ciglitazone, darglitazone, englitazone, isaglitazone, pioglitazone, rosiglitazone and troglitazone), a PPAR α/γ agonist (e.g., CLX-0940, GW-1536, GW-1929, GW-2433, KRP-297, L-796449, LR-90, MK-0767 and SB-219994), a biguanide (e.g., metformin), a glucagon-like peptide 1 (GLP-1) agonist (e.g., exendin-3 and exendin-4), a protein tyrosine phosphatase-1B (PTP-1B) inhibitor (e.g., trodusquemine, hyrtiosal extract, and compounds disclosed by Zhang, S., et al., Drug Discovery Today, 12(9/10), 373-381 (2007)), SIRT-1 inhibitor (e.g., reservatrol), a dipeptidyl peptidease IV (DPP-IV) inhibitor (e.g., sitagliptin, vildagliptin, alogliptin and saxagliptin), an insulin secreatagogue, a fatty acid oxidation inhibitor, an A2 antagonist, a c-jun amino-terminal kinase (JNK) inhibitor, insulin, an insulin mimetic, a glycogen phosphorylase inhibitor, a VPAC2 receptor agonist, and a SGLT2 inhibitor (sodium dependent glucose transporter inhibitors such as dapagliflozin, etc). Preferred anti-diabetic agents are metformin and DPP-IV inhibitors (e.g., sitagliptin, vildagliptin, alogliptin and saxagliptin).

Suitable anti-obesity agents include 11β-hydroxy steroid dehydrogenase-1 (11β-HSD type 1) inhibitors, stearoyl-CoA desaturase-1 (SCD-1) inhibitor, MCR-4 agonists, cholecystokinin-A (CCK-A) agonists, monoamine reuptake inhibitors (such as sibutramine), sympathomimetic agents, β₃ adrenergic agonists, dopamine agonists (such as bromocriptine), melanocyte-stimulating hormone analogs, 5HT2c agonists, melanin concentrating hormone antagonists, leptin (the OB protein), leptin analogs, leptin agonists, galanin antagonists, lipase inhibitors (such as tetrahydrolipstatin, i.e. orlistat), anorectic agents (such as a bombesin agonist), neuropeptide-Y antagonists (e.g., NPY Y5 antagonists), PYY₃₋₃₆(including analogs thereof), thyromimetic agents, dehydroepiandrosterone or an analog thereof, glucocorticoid agonists or antagonists, orexin antagonists, glucagon-like peptide-1 agonists, ciliary neurotrophic factors (such as Axokine™ available from Regeneron Pharmaceuticals, Inc., Tarrytown, N.Y. and Procter & Gamble Company, Cincinnati, Ohio), human agouti-related protein (AGRP) inhibitors, ghrelin antagonists, histamine 3 antagonists or inverse agonists, neuromedin U agonists, MTP/ApoB inhibitors (e.g., gut-selective MTP inhibitors, such as dirlotapide), opioid antagonist, orexin antagonist, and the like.

Preferred anti-obesity agents for use in the combination aspects of the present invention include gut-selective MTP inhibitors (e.g., dirlotapide, mitratapide and implitapide, R56918 (CAS No. 403987) and CAS No. 913541-47-6), CCKa agonists (e.g., N-benzyl-2-[4-(1H-indol-3-ylmethyl)-5-oxo-1-phenyl-4,5-dihydro-2,3,6,10b-tetraaza-benzo[e]azulen-6-yl]-N-isopropyl-acetamide described in PCT Publication No. WO 2005/116034 or US Publication No. 2005-0267100 A1), 5HT2c agonists (e.g., lorcaserin), MCR4 agonist (e.g., compounds described in U.S. Pat. No. 6,818,658), lipase inhibitor (e.g., Cetilistat), PYY₃₋₃₆(as used herein “PYY₃₋₃₆” includes analogs, such as peglated PYY₃₋₃₆ e.g., those described in US Publication 2006/0178501), opioid antagonists (e.g., naltrexone), oleoyl-estrone (CAS No. 180003-17-2), obinepitide (TM30338), pramlintide (Symlin®), tesofensine (NS2330), leptin, liraglutide, bromocriptine, orlistat, exenatide (Byetta®), AOD-9604 (CAS No. 221231-10-3) and sibutramine. Preferably, compounds of the present invention and combination therapies are administered in conjunction with exercise and a sensible diet.

All of the above recited U.S. patents and publications are incorporated herein by reference.

### Pharmaceutical Formulations

The present invention also provides pharmaceutical compositions which comprise a therapeutically effective amount of a compound, or a pharmaceutically acceptable salt thereof, in admixture with at least one pharmaceutically acceptable excipient. The compositions include those in a form adapted for oral, topical or parenteral use and can be used for the treatment of diabetes and related conditions as described above.

The composition can be formulated for administration by any route known in the art, such as subdermal, inhalation, oral, topical, parenteral, etc. The compositions may be in any form known in the art, including but not limited to tablets, capsules, powders, granules, lozenges, or liquid preparations, such as oral or sterile parenteral solutions or suspensions.

Tablets and capsules for oral administration may be in unit dose presentation form, and may contain conventional excipients such as binding agents, for example syrup, acacia, gelatin, sorbitol, tragacanth, or polyvinylpyrollidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice.

Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerin, propylene glycol, or ethyl alcohol; preservatives, for example methyl or propyl p-hydroxybenzoate or sorbic acid, and, if desired, conventional flavoring or coloring agents.

For parenteral administration, fluid unit dosage forms are prepared utilizing the compound and a sterile vehicle, water being preferred. The compound, depending on the vehicle and concentration used, can be either suspended or dissolved in the vehicle or other suitable solvent. In preparing solutions, the compound can be dissolved in water for injection and filter sterilized before filling into a suitable vial or ampoule and sealing. Advantageously, agents such as local anesthetics, preservatives and buffering agents etc. can be dissolved in the vehicle. To enhance the stability, the composition can be frozen after filling into the vial and the water removed under vacuum. The dry lyophilized powder is then sealed in the vial and an accompanying vial of water for injection may be supplied to reconstitute the liquid prior to use. Parenteral suspensions are prepared in substantially the same manner except that the compound is suspended in the vehicle instead of being dissolved and sterilization cannot be accomplished by filtration. The compound can be sterilized by exposure to ethylene oxide before suspending in the sterile vehicle. Advantageously, a surfactant or wetting agent is included in the composition to facilitate uniform distribution of the compound.

The compositions may contain, for example, from about 0.1% to about 99 by weight, of the active material, depending on the method of administration. Where the compositions comprise dosage units, each unit will contain, for example, from about 0.1 to 900 mg of the active ingredient, more typically from 1 mg to 250 mg.

Compounds of the invention can be formulated for administration in any convenient way for use in human or veterinary medicine, by analogy with other anti-diabetic agents. Such methods are known in the art and have been summarized above. For a more detailed discussion regarding the preparation of such formulations; the reader's attention is directed to Remington's Pharmaceutical Sciences, 21^{st} Edition, by University of the Sciences in Philadelphia.

Embodiments of the present invention are illustrated by the following Examples. It is to be understood, however, that the embodiments of the invention are not limited to the specific details of these Examples, as other variations thereof will be known, or apparent in light of the instant disclosure, to one of ordinary skill in the art.

### EXAMPLES

Unless specified otherwise, starting materials are generally available from commercial sources such as Aldrich Chemicals Co. (Milwaukee, Wis.), Lancaster Synthesis, Inc. (Windham, N.H.), Acros Organics (Fairlawn, N.J.), Maybridge Chemical Company, Ltd. (Cornwall, England), Tyger Scientific (Princeton, N.J.), and AstraZeneca Pharmaceuticals (London, England), Mallinckrodt Baker (Phillipsburg N.J.); EMD (Gibbstown, N.J.)

### General Experimental Procedures

### General Experimental Procedures

NMR spectra were recorded on a Varian Unity™ 400 (DG400-5 probe) or 500 (DG500-5 probe—both available from Varian Inc., Palo Alto, Calif.) at room temperature at 400 MHz or 500 MHz respectively for proton analysis. Chemical shifts are expressed in parts per million (delta) relative to residual solvent as an internal reference. The peak shapes are denoted as follows: s, singlet; d, doublet; dd, doublet of doublet; t, triplet; q, quartet; m, multiplet; bs, broad singlet; 2s, two singlets.

Atmospheric pressure chemical ionization mass spectra (APCI) were obtained on a Waters™ Spectrometer (Micromass ZMD, carrier gas: nitrogen) (available from Waters Corp., Milford, Mass., USA) with a flow rate of 0.3 mL/minute and utilizing a 50:50 water/acetonitrile eluent system. Electrospray ionization mass spectra (ES) were obtained on a liquid chromatography mass spectrometer from Waters™ (Micromass ZQ or ZMD instrument (carrier gas: nitrogen) (Waters Corp., Milford, Mass., USA) utilizing a gradient of 95:5-0:100 water in acetonitrile with 0.01% formic acid added to each solvent. These instruments utilized a Varian Polaris 5 C18-A20×2.0 mm column (Varian Inc., Palo Alto, Calif.) at flow rates of 1 mL/minute for 3.75 minutes or 2 mL/minute for 1.95 minutes.

Column chromatography was performed using silica gel with either Flash 40 Biotage™ columns (ISC, Inc., Shelton, Conn.) or Biotage™ SNAP cartridge KPsil or Redisep Rf silica (from Teledyne Isco Inc) under nitrogen pressure.

Concentration in vacuo refers to evaporation of solvent under reduced pressure using a rotary evaporator.

### Pharmacological Data

The practice of the present invention for the treatment of diseases modulated by the agonist activation of the G-protein-coupled receptor GPR119 with compounds of the present invention can be evidenced by activity in at least one of the protocols described herein below. The source of supply is provided in parenthesis.

### In-Vitro Assay

The assay for GPR119 agonists utilizes a cell-based (hGPR119 HEK293-CRE β-lactamase) reporter construct where agonist activation of human GPR119 is coupled to β-lactamase production via a cyclic AMP response element (CRE). GPR119 activity is then measured utilizing a FRET-enabled R-lactamase substrate, CCF4-AM (Live Blazer FRET-B/G Loading kit, Invitrogen cat #K1027). Specifically, hGPR119-HEK-CRE-β-lactamase cells (Invitrogen 2.5×10⁷/mL) were removed from liquid nitrogen storage, and diluted in plating medium (Dulbecco's modified Eagle medium high glucose (DMEM; Gibco Cat #11995-065), 10% heat inactivated fetal bovine serum (HIFBS; Sigma Cat #F4135), 1× MEM Nonessential amino acids (Gibco Cat #15630-080), 25 mM HEPES pH 7.0 (Gibco Cat #15630-080), 200 nM potassium clavulanate (Sigma Cat #P3494). The cell concentration was adjusted using cell plating medium and 50 μL of this cell suspension (12.5×10⁴ viablecells) was added into each well of a black, clear bottom, poly-d-lysine coated 384-well plate (Greiner Bio-One cat #781946) and incubated at 37° C. in a humidified environment containing 5% carbon dioxide. After 4 hours the plating medium was removed and replaced with 40 μL of assay medium (Assay medium is plating medium without potassium clavulanate and HIFBS). Varying concentrations of each compound to be tested was then added in a volume of 10 uL (final DMSO≦0.5%) and the cells were incubated for 16 hours at 37° C. in a humidified environment containing 5% carbon dioxide. Plates were removed from the incubator and allowed to equilibrate to room temperature for approximately 15 minutes. 10 uL of 6× CCF4/AM working dye solution (prepared according to instructions in the Live Blazer FRET-B/G Loading kit, Invitrogen cat #K1027) was added per well and incubated at room temperature for 2 hours in the dark. Fluorescence was measured on an EnVision fluorimetric plate reader, excitation 405 nm, emission 460 nm/535 nm. EC₅₀ determinations were made from agonist-response curves analyzed with a curve fitting program using a 4-parameter logistic dose-response equation.

The following results were obtained:

| Example | Run | EC₅₀ (nM) | Intrinsic Activity* (%) |
|---|---|---|---|
| 1 | 1 | 10.5 | 90 |
| 1 | 2 | 12.8 | 88 |
| 1 | 3 | 23.3 | 87 |
| 2 | 1 | 23.8 | 93 |

*The intrinsic activity is the percent of maximal activity of the test compound, relative to the activity of a standard GPR119 agonist, 4-[[6-[(2-fluoro-4 methylsulfonylphenyl)amino]pyrimidin-4-yl]oxy]piperidine-1-carboxylic acid isopropyl ester (WO2005121121), at a final concentration of 10 μM.

### Preparation of Starting Materials

### Preparation #1

Scheme A illustrates the preparation of syn and anti 9-hydroxy-3-oxa-7-azabicyclo[3.3.1]nonane-7-carboxylate. The experimental details are described in detail below.

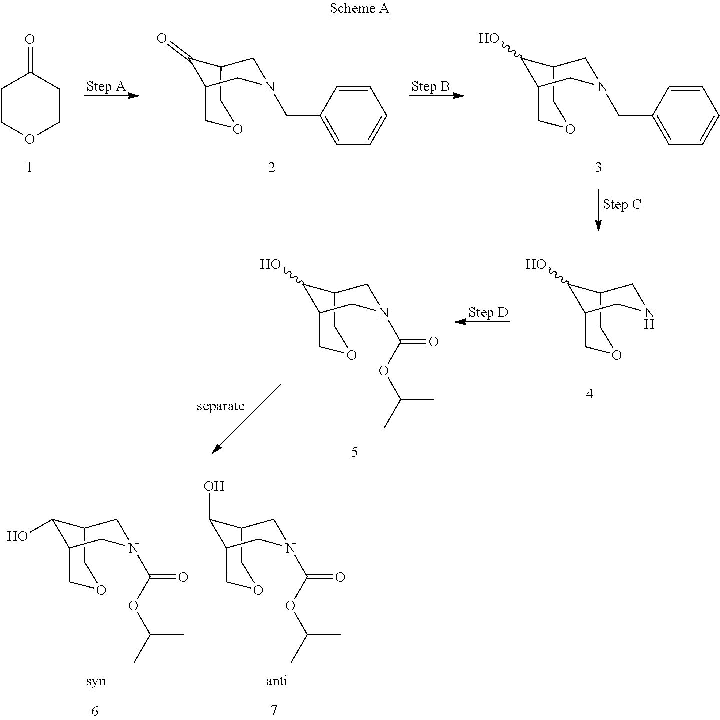

Step A. Synthesis of 7-benzyl-3-oxa-7-azabicyclo[3.3.1]nonan-9-one-hydrochloride salt (2): A solution of tetrahydro-4H-pyran-4-one 1 (60.0 g, 0.60 mol), benzylamine (63.4 g, 0.60 mol) and glacial acetic acid (35.9 g, 0.60 mol) in dry methanol (1.2 L) was added to a stirred suspension of paraformaldehyde (39.6 g, 1.3 mol) in dry methanol (1.2 L) over a period of 75 minutes at 65° C. A second portion of paraformaldehyde (39.6 g, 1.3 mol) was added, and the mixture was stirred for 1 hour at 65° C. The reaction was quenched with water (1.2 L) and 1 M aqueous potassium hydroxide solution (600 mL). The mixture was extracted with ethyl acetate (3 L×3). The combined organic layers were dried over sodium sulfate, filtered and the filtrate was concentrated to dryness in vacuo. The residue was purified by column chromatography (petroleum ether/ethyl acetate=20:1˜2:1) to afford a brown oil. The residue was diluted with 6 M anhydrous hydrochloric acid in dioxane (500 mL), and the mixture was stirred for 30 minutes at room temperature. The solvent was removed in vacuo, and acetone (500 mL) was added. The resulting mixture was sonicated at room temperature for 30 minutes causing a white precipitate to form. The mixture was filtered, and the solid was washed with acetone and then dried under vacuum to afford the desired product as a white solid (21 g, 13%): ¹H NMR (400 MHz, D₂O) delta 7.43-7.42 (m, 5H), 4.66 (s, 2H), 3.95-3.90 (m, 4H), 3.54-3.47 (m, 4H); 1.96 (bs, 2H); MS (ES+): 232.0 (M+1).

Step B. Synthesis of 7-benzyl-3-oxa-7-azabicyclo[3.3.1]nonan-9-ol (mixture of syn and anti-isomers) (3): 7-benzyl-3-oxa-7-azabicyclo[3.3.1]nonan-9-one hydrochloride salt (4.40 g, 16.9 mmol) was suspended in ethanol (40 mL) and anhydrous tetrahydrofuran (40 mL). The mixture was cooled with an ice bath, and sodium borohydride (1.5 g, 37.3 mmol) was added in one portion. The mixture was allowed to warm slowly over 4 hours to room temperature. The reaction was then concentrated in vacuo to remove most of the ethanol and tetrahydrofuran. The mixture was partitioned between methyl tert-butyl ether and aqueous 1.0 M sodium hydroxide solution. The solution was stirred for 30 minutes followed by separation of the two layers. The aqueous layer was extracted with methyl tert-butyl ether. The organic extracts were combined, washed with brine, and dried over sodium sulfate. The mixture was filtered and the filtrate was concentrated in vacuo to give a clear oil, which partially solidified on standing to an oily white solid (3.71 g, 94%). This mixture of syn and anti 7-benzyl-3-oxa-7-azabicyclo[3.3.1]nonan-9-ol isomers was used in the next step without further purification. MS (ES+): 234.1 (M+H).

Step C. Synthesis of 3-oxa-7-azabicyclo[3.3.1]nonan-9-ol (mixture of syn and anti-isomers) (4): The starting mixture of syn and anti 7-benzyl-3-oxa-7-azabicyclo[3.3.1]nonan-9-ol isomers (3.71 g, 15.9 mmol) was dissolved in ethanol (120 mL), and Pd(OH)₂ (450 mg) was added. The mixture was shaken for 2.5 hours under 50 psi of hydrogen in a Parr shaker. The mixture was filtered through Celite (registered trademark), and the collected solid was washed three times with methanol. The filtrate was concentrated in vacuo to give an oily solid. This oily solid was dissolved in ethyl acetate and heptane was added. The solution was concentrated in vacuo to give a mixture of syn and anti isomers of 3-oxa-7-azabicyclo[3.3.1]nonan-9-ol as a white solid (2.08 g, 91%). This material was used in the next step without further purification. MS (ES+): 144.1 (M+H).

Step D. Synthesis of isopropyl 9-hydroxy-3-oxa-7-azabicyclo[3.3.1]nonane-7-carboxylate (mixture of syn and anti-isomers) (5): To a dichloromethane (15 mL) solution of the mixture of syn and anti isomers of 3-oxa-7-azabicyclo[3.3.1]nonan-9-ol (2.08 g, 14.5 mmol) and diisopropylethylamine (2.80 mL, 16.0 mmol) at 0° C. was added isopropyl chloroformate (14.2 mL, 14.2 mmol, 1.0 M in toluene) dropwise. The reaction mixture was allowed to warm to room temperature over 14 hours. The reaction was then diluted with aqueous 1 M hydrochloric acid (50 mL), and the aqueous layer separated. The organic layer was washed sequentially with water (50 mL) and brine (50 mL) and then dried over sodium sulfate. The mixture was filtered, and the filtrate was concentrated in vacuo to give a colorless oil. This oil was dissolved in ethyl acetate; heptane was added and the mixture was concentrated. The resulting oil was dried under vacuum to give the mixture of syn and anti isomers of isopropyl 9-hydroxy-3-oxa-7-azabicyclo[3.3.1]nonane-7-carboxylate as a clear oil (2.74 g, 82%). MS (ES+): 230.1 (M+H).

Step E. Separation of the syn and anti-isomers of isopropyl 9-hydroxy-3-oxa-7-azabicyclo[3.3.1]nonane-7-carboxylate: A mixture of syn and anti isomers of isopropyl 9-hydroxy-3-oxa-7-azabicyclo[3.3.1]nonane-7-carboxylate (5.04 g, 35.1 mmol) was separated via preparatory high pressure liquid chromatography utilizing a Chiralpak AD-H column (21×250 mm) with mobile phase of 85:15 carbon dioxide and methanol respectively at a flow rate of 65 mL/minute. The wavelength for monitoring the separation was 210 nm. The analytical purity of each isomer was determined using analytical high pressure chromatography using a Chiralpak AD-H (4.6 mm×25 cm) column with a mobile phase of 85:15 carbon dioxide and methanol respectively at a flow rate of 2.5 mL/minute. The wavelength for monitoring the peaks was 210 nm. The following two isomers were obtained:

9-syn-hydroxy-3-oxa-7-azabicyclo[3.3.1]nonane-7-carboxylate (6) (1.34 g): clear oil which solidified on standing, Retention time (Rₜ)=2.3 minutes, ¹H NMR (400 MHz, DMSO-d₆,): delta 5.12 (d, 1H, J=2.8 Hz), 4.76-4.71 (m, 1H), 4.20 (d, 1H, J=13 Hz), 4.16 (d, 1H, J=13 Hz), 3.96-3.92 (m, 3H), 3.79 (d, 1H, J=3 Hz), 3.55 (s, 1H), 3.52 (S, 1H), 3.08 (d, 1H, J=13 Hz), 2.98 (d, 1H, J=13 Hz), 1.47 (m, 2H) 1.16 (d, 3H, J=3 Hz), 1.15 (d, 3H, J=3 Hz); MS (ES+): 230.2 (M+H).

9-anti-hydroxy-3-oxa-7-azabicyclo[3.3.1]nonane-7-carboxylate (7) (1.70 g): amber oil, Rₜ=3.08 minutes, ¹H NMR (400 MHz, DMSO-d₆): delta 5.11 (d, 1H, J=2.8 Hz), 4.74-4.67 (m, 1H), 3.89 (d, 1H, J=13 Hz), 3.84-3.78 (m, 3H, J=11 Hz), 3.80 (d, 1H, J=6 Hz), 3.78 (d, 1H, J=3 Hz), 3.52-3.47 (m, 2H), 3.35-3.30 (m, 1H), 3.24-3.20 (m, 1H), 1.53 (s, 1H), 1.51 (s, 1H), 1.13 (d, 3H, J=1 Hz), 1.16 (d, 3H, J=1 Hz); MS (ES+): 230.2 (M)

### EXAMPLES

### Example I

### Isopropyl 9-syn-({7-[2-fluoro-4-(methylsulfonyl)phenyl]-6,7-dihydro-5H-pyrrolo[2,3-d]pyrimidin-4-yl}oxy)-3-oxa-7-azabicyclo[3.3.1]nonane-7-carboxylate

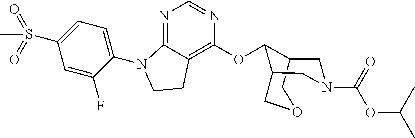

The known compound 4-chloro-7-(2-fluoro-4-(methylsulfonyl)phenyl)-6,7-dihydro-5H-pyrrolo[2,3-d]pyrimidine (WO 2008/008895) (45 mg, 0.14 mmol) and 9-syn-hydroxy-3-oxa-7-azabicyclo[3.3.1]nonane-7-carboxylate (33 mg, 0.14 mmol) were placed in a vial and anhydrous dioxane (1 mL) was added. The mixture was heated at 95° C. under a nitrogen atmosphere. A solution of 1M sodium bis(trimethylsilyl)amide in toluene (0.192 mL, 0.192 mmol) was then added. The reaction mixture was stirred at 95° C. for 2 hours. The reaction mixture was cooled to room temperature and then was diluted with ethyl acetate (30 mL) and water (20 mL). The organic phase was washed with aqueous sodium bicarbonate. The aqueous phase was extracted two times with ethyl acetate. The combined organic layers were dried over magnesium sulfate, filtered and the filtrate was evaporated. The crude material was purified by column chromatography using a gradient of 0-6% dichloromethane/methanol to give isopropyl 9-syn-({7-[2-fluoro-4-(methylsulfonyl)phenyl]-6,7-dihydro-5H-pyrrolo[2,3-d]pyrimidin-4-yl}oxy)-3-oxa-7-azabicyclo[3.3.1]nonane-7-carboxylate as a white solid 45 mg (64%). LCMS: 521.0 (M+1). 1H NMR (400 MHz, deuterochloroform) delta 1.24 (d, J=6.25 Hz, 6H) 1.92 (d, J=13.27 Hz, 2H) 3.04 (s, 3H) 3.12-3.25 (m, 3H) 3.29 (d, J=13.86 Hz, 1H) 3.83 (d, J=11.71 Hz, 1H) 3.91 (d, J=11.32 Hz, 1H) 4.08 (t, J=10.93 Hz, 2H) 4.17-4.27 (m, 2H) 4.45 (d, J=13.86 Hz, 1H) 4.61 (d, J=13.86 Hz, 1H) 4.90-5.00 (m, 1H) 5.36 (t, J=3.51 Hz, 1H) 7.67-7.75 (m, 2H) 8.03 (dd, J=8.78, 7.42 Hz, 1H) 8.26 (s, 1H).

### Example 2

### Isopropyl 9-anti-({7-[2-fluoro-4-(methylsulfonyl)phenyl]-6,7-dihydro-5H-pyrrolo[2,3-d]pyrimidin-4-yl}oxy)-3-oxa-7-azabicyclo[3.3.1]nonane-7-carboxylate

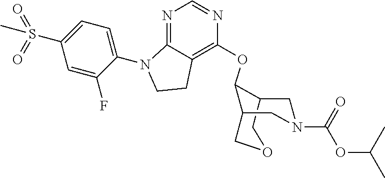

4-chloro-7-(2-fluoro-4-(methylsulfonyl)phenyl)-6,7-dihydro-5H-pyrrolo[2,3-d]pyrimidine (42 mg, 0.13 mmol) and 9-anti-hydroxy-3-oxa-7-azabicyclo[3.3.1]nonane-7-carboxylate (29.5 mg, 0.13 mmol) were placed in a vial and anhydrous dioxane (1 mL) was added. The mixture was heated at 95° C. under a nitrogen atmosphere. A solution of 1M sodium bis(trimethylsilyl)amide in toluene (0.18 mL, 0.18 mmol) was then added. The reaction mixture was stirred at 95° C. for 2 hours. The reaction mixture was cooled to room temperature and then was diluted with ethyl acetate (30 mL) and water (20 mL). The organic phase was washed with aqueous sodium bicarbonate. The aqueous phase was extracted two times with ethyl acetate. The combined organic layers were dried over magnesium sulfate, filtered and the filtrate was evaporated. The crude material was purified by column chromatography using a gradient of 0-6% dichloromethane/methanol to give 9-anti-({7-[2-fluoro-4-(methylsulfonyl)phenyl]-6,7-dihydro-5H-pyrrolo[2,3-d]pyrimidin-4-yl}oxy)-3-oxa-7-azabicyclo[3.3.1]nonane-7-carboxylate as a white solid 45 mg (68%). LCMS: 521.2 (M+1).

1H NMR (400 MHz, deuterochloroform) delta 1.23 (dd, J=6.34, 2.64 Hz, 6H) 1.93-2.06 (m, 2H) 3.03 (s, 3H) 3.14 (t, J=8.69 Hz, 2H) 3.33-3.50 (m, 2H) 3.81(t, J=9.57 Hz, 2H) 4.05-4.34 (m, 6H) 4.89-5.02 (m, 1H) 5.41 (t, J=3.61 Hz, 1H) 7.65-7.74 (m, 2H) 8.02 (dd, J=8.78, 7.42 Hz, 1H) 8.25 (s, 1H)

What is claimed is:

1. A compound of the formula: 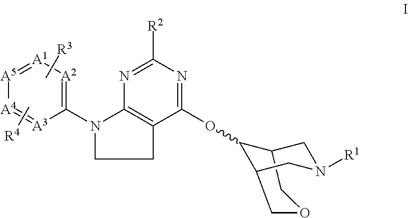
in which
R¹ is represented by C(O)—O—R⁵ or a substituted pyrimidine represented by the following formula: 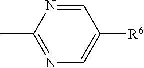
R² is represented by hydrogen or methyl;
R³ is represented by a subsituent selected from the group consisting of hydrogen, cyano, halogen, CF₃, OCF₃, C₁-C₅ alkoxy, and C₁-C₅ alkyl;
R⁴ is absent, or is represented by SO₂—R⁷, CO—NR⁸R⁹, tetrazole, C₁-C₅ alkyl, NH₂, —NH—C₁-C₅ alkyl, —NH—CO—C₁-C₅ alkyl, —NH—(CH₂)₂—OH;
R⁵ is represented by C₁-C₅ alkyl, or C₃-C₆ cycloalkyl;
R⁶ is represented by CF₃, C₁-C₅ alkyl, halogen, cyano, or C₃-C₆ cycloalkyl;
R⁷ is represented by C₁-C₅ alkyl, —NH₂, or (CH₂)₂—OH, C₃-C₆ cycloalkyl;
R⁸ is represented by hydrogen or C₁-C₅ alkyl,
R⁹ is represented by hydrogen, C₁-C₅ alkyl, C₃-C₆ cycloalkyl, CH₂—CH₂—OH, CH₂—CH₂—O—CH₃, CH₂—CH₂—CH₂—O—CH₃, CH₂—CH₂—CH₂—OH, 3-oxetanyl, 3-hydroxycyclobutyl,
A¹, A², A³, A⁴, and A⁵ are each independently represented by CH, N-oxide, or N;
with the proviso that:
a) no more than 2 of A¹, A², A³, A⁴, and A⁵ are represented by N;
b) no more than 1 of A¹, A², A³, A⁴, and A⁵ are represented by N-oxide;
or a pharmaceutically acceptable salt thereof.

2. A compound according to claim 1 in which A¹-A⁵ forms a phenyl ring.

3. A compound according to claim 1 in which A¹-A⁵ forms a pyridyl ring.

4. A compound according to claim 2 in which R⁴ is SO₂—R⁷.

5. A compound according to claim 4 in which R³ is fluoro or hydrogen.

6. A compound according to claim 5 in which R² is hydrogen.

7. A compound according to claim 6 in which R¹ is —C(O)—O—C₁-C₅ alkyl.

8. Isopropyl 9-anti-({7-[2-fluoro-4-(methylsulfonyl)phenyl]-6,7-dihydro-5H-pyrrolo[2,3-d]pyrimidin-4-yl}oxy)-3-oxa-7-azabicyclo[3.3.1]nonane-7-carboxylate, a pharmaceutically acceptable salt thereof, or a pharmaceutically acceptable co-crystal thereof.

9. Isopropyl 9-syn-({7-[2-fluoro-4-(methylsulfonyl)phenyl]-6,7-dihydro-5H-pyrrolo[2,3-d]pyrimidin-4-yl}oxy)-3-oxa-7-azabicyclo[3.3.1]nonane-7-carboxylate, a pharmaceutically acceptable salt thereof, or a pharmaceutically acceptable co-crystal thereof.

10. A pharmaceutical composition comprising a compound according to claim 1, present in a therapeutically effective amount, in admixture with at least one pharmaceutically acceptable excipient.

11. The composition of claim 10 further comprising at least one additional pharmaceutical agent selected from the group consisting of an anti-obesity agent and an anti-diabetic agent.

12. The composition of claim 11 wherein said anti-obesity agent is selected from the group consisting of dirlotapide, mitratapide, implitapide, R56918 (CAS No. 403987), CAS No. 913541-47-6, lorcaserin, cetilistat, PYY_{3-36,} naltrexone, oleoyl-estrone, obinepitide, pramlintide, tesofensine, leptin, liraglutide, bromocriptine, orlistat, exenatide, AOD-9604 (CAS No. 221231-10-3) and sibutramine.

13. The composition of claim 11 wherein said anti-diabetic agent is selected from the group consisting of metformin, acetohexamide, chlorpropamide, diabinese, glibenclamide, glipizide, glyburide, glimepiride, gliclazide, glipentide, gliquidone, glisolamide, tolazamide, tolbutamide, tendamistat, trestatin, acarbose, adiposine, camiglibose, emiglitate, miglitol, voglibose, pradimicin-Q, salbostatin, balaglitazone, ciglitazone, darglitazone, englitazone, isaglitazone, pioglitazone, rosiglitazone, troglitazone, exendin-3, exendin-4, trodusquemine, reservatrol, hyrtiosal extract, sitagliptin, vildagliptin, alogliptin and saxagliptin.

14. A method for the treatment of diabetes comprising the administration of an effective amount of compound according to claim 1 to a patient in need thereof.

15. A method for treating a metabolic or metabolic-related disease, condition or disorder comprising the step of administering to a patient a therapeutically effective amount of a compound of claim 1.

16. A method for treating a condition selected from the group consisting of hyperlipidemia, type I diabetes, type II diabetes mellitus, idiopathic type I diabetes (Type Ib), latent autoimmune diabetes in adults (LADA), early-onset type 2 diabetes (EOD), youth-onset atypical diabetes (YOAD), maturity onset diabetes of the young (MODY), malnutrition-related diabetes, gestational diabetes, coronary heart disease, ischemic stroke, restenosis after angioplasty, peripheral vascular disease, intermittent claudication, myocardial infarction (e.g. necrosis and apoptosis), dyslipidemia, post-prandial lipemia, conditions of impaired glucose tolerance (IGT), conditions of impaired fasting plasma glucose, metabolic acidosis, ketosis, arthritis, obesity, osteoporosis, hypertension, congestive heart failure, left ventricular hypertrophy, peripheral arterial disease, diabetic retinopathy, macular degeneration, cataract, diabetic nephropathy, glomerulosclerosis, chronic renal failure, diabetic neuropathy, metabolic syndrome, syndrome X, premenstrual syndrome, coronary heart disease, angina pectoris, thrombosis, atherosclerosis, myocardial infarction, transient ischemic attacks, stroke, vascular restenosis, hyperglycemia, hyperinsulinemia, hyperlipidemia, hypertrygliceridemia, insulin resistance, impaired glucose metabolism, conditions of impaired glucose tolerance, conditions of impaired fasting plasma glucose, obesity, erectile dysfunction, skin and connective tissue disorders, foot ulcerations and ulcerative colitis, endothelial dysfunction and impaired vascular compliance, hyper apo B lipoproteinemia, Alzheimer's, schizophrenia, impaired cognition, inflammatory bowel disease, ulcerative colitis, Crohn's disease, and irritable bowel syndrome,comprising the administration of an effective amount of a compound according to claim 1.

17. A method for treating a metabolic or metabolic-related disease, condition or disorder comprising the step of administering to a patient in need of such treatment two separate pharmaceutical compositions comprising
(ii) a second composition comprising at least one additional pharmaceutical agent selected from the group consisting of an anti-obesity agent and an anti-diabetic agent, and at least one pharmaceutically acceptable excipient.

18. The method of claims 16 wherein said first composition and said second composition are administered simultaneously.

19. The method of claim 16 wherein said first composition and said second composition are administered sequentially and in any order.

19. (canceled)

20. (canceled)
